(19) United States
(12) Patent Application Publication
(10) Pub. No.: US 2020/0069966 A1
(43) Pub. Date: Mar. 5, 2020

(54) Neuro-Training Device, System and Method of Use

(71) Applicant: Excel Management, LLC d/b/a BrainTap Technologies, New Bern, NC (US)

(72) Inventor: Patrick K. Porter, New Bern, NC (US)

(73) Assignee: Excel Management, LLC d/b/a BrainTap Technologies, New Bern, NC (US)

(21) Appl. No.: 16/665,213

(22) Filed: Oct. 28, 2019

Related U.S. Application Data
(60) Provisional application No. 62/723,658, filed on Aug. 28, 2018.

(51) Int. Cl. A61N 5/06; H04R 5/04; H04R 5/033; G02B 27/01; G09G 3/22

(52) U.S. Cl. CPC A61N 5/0622; H04R 5/04; H04R 5/033; A61N 2005/0652; G09G 3/22; A61N 2005/0648; G02B 27/0172

(57) ABSTRACT

Embodiments of the invention are directed towards neuro-training devices, systems and methods of use thereof that utilize encoded light and audio signals, singly or in combination, to stimulate the human brain and synchronize brain wave function. Various embodiments further comprise novel auriculotherapy methods. Embodiments of the neuro-training invention generally comprise a human interface device component(s), an electronic file playback device component, and one or more audio/visual (A/V) files for playback by the playback device component. Light and/or audio signals encoded in the A/V files are run (played) by the playback device component and transmitted to the human interface device component(s). The resulting audio and/or light signals received by the human interface device component(s) and the audio and light transmitted and emitted therefrom and received by the user's eyes and ears maximize neuroplasticity—the brain's ability to reorganize itself by forming new neural connections, resulting in greater brain flexibility and resiliency.

### CROSS-REFERENCES TO RELATED APPLICATIONS

This application is the Non-Provisional Application of Provisional Application No. 62/723,658 (Confirmation No. 8502) filed on Aug. 28, 2018 for “Auriculotherapy Apparatus and System and Methods of Use Thereof” by Patrick K. Porter, PhD. This Non-Provisional Application claims priority to and the benefit of that Provisional Application, the contents and subject of which are incorporated herein by reference in their entirety, including all references cited and incorporated within the Provisional Application.

### STATEMENT REGARDING FEDERALLY SPONSORED RESEARCH OR DEVELOPMENT

Not Applicable

### SUMMARY OF THE INVENTION

Embodiments of the invention are directed towards neuro-training devices, systems and methods of use thereof that utilize encoded light and audio signals, singly or in combination, to stimulate the human brain and synchronize brain wave function. Various embodiments further comprise novel auriculotherapy methods.

Embodiments of the neuro-training invention generally comprise a human interface device portion, an electronic file playback device portion, and one or more audio/visual files (collectively, “A/V files”) for playback by the playback device portion. The A/V files are run (played) by the playback device portion thereby generating associated audio/visual signals for transmission by the playback device for receipt by the human interface device portion. The human interface device, which is worn by a user, generally comprises: 1) one or more light emission features V, such as, for example, one or more lights such as LEDs, for converting electrical signals transmitted by the playback device to corresponding light emissions generally in the form of light pulses (collectively, all such electrical light/visual signals referred to as “light signals,”), and/or 2) one or more electroacoustic converters, such as audio electroacoustic transducers (e.g., audio speakers), for converting electrical audio signals transmitted by the playback device into corresponding audio sounds (collectively, all such electrical audio signals referred to as “audio signals”) (collectively, the light signals and the audio signals referred to herein as “A/V signals”).

The human interface device portion, which may comprise a separate audio (ear) stimulation component (“audio transmission component”) and visual (eye) stimulation component (“visual display component”), converts the respective audio signals to sound for receipt and detection by the ear(s) of a user thereof and the light signals to light emissions—typically pulsating light as encoded in the respective A/V file—for receipt and detection by the ear(s) and/or eye(s) of a user thereof depending on the respective embodiment.

In embodiments, the A/V signals received by the human interface device are generated by and transmitted from the electronic playback device, said playback device being capable of accessing and running (playing) the A/V files, which comprise various pre-recorded digital audio and/or visual files stored on the playback device or otherwise stored on such other digital storage media, including storage media comprising the internet (e.g., servers comprising the “cloud”) and accessible by the playback device for playback and transmission of the A/V signal(s) of the corresponding A/V file(s) to the human interface device. Alternatively, in embodiments, the A/V files are stored online in the cloud/internet, accessed by a user via an online platform through the playback device, and streamed to the playback device through a functional network connection for immediate playback and transmission to the human interface device portion(s).

In embodiments, the playback device and the human interface device may be integrated into a single functional component or device that is worn by a user. The human interface feature of the single component may comprise a visual display component, an audio transmission component, or both—a visual display component and an audio transmission component.

In various embodiments, the pre-encoded light signals may be embedded in an audio file, such as, for example, an MP3 file, whereupon when the MP3 file is run or played by the playback device, at various pre-determined times as encoded in the MP3 file, the file generates a specific light or electric signals to be converted to light as described herein. As such, A/V files, such as MP3 files or any other commonly used multimedia files, may comprise one or more audio signal generating component(s) and one or more light signal generating component(s), which are synchronously encoded in the file to simultaneously generate specific audio signals and light signals to achieve the desired effect on brain stimulation and brain wave synchronization.

The human interface device generally comprises an audio transmission component and a visual display component. The audio transmission component may comprise a wearable headphone type device for covering one or both ears of a user, wherein said headphone covering the ear(s) may comprise one or more audio speaker(s) (or any such other electroacoustic converter for converting the audio signal(s) to sound) for the transmission of sound from the corresponding audio signal or audio signal portion of the A/V signal to the user's ear(s). In other embodiments, the audio transmission component, e.g., headphone set, may further comprise a light emission feature comprising one or more lights, such as, for example, LED lights, that emit light and/or light pulses in various wavelength frequencies of visible and/or non-visible light from the electromagnetic spectrum in accordance with the corresponding electrical light signal or light signal portion of the A/V signal to the auricle of the ear(s) and according to the wavelength frequency of the associated LED light triggered by the light signal. In yet further embodiments, the audio transmission component portion may comprise more simplistic headphones, earphones, earbuds, air buds, or any other commonly known and used human audio transmission devices for the transmission of audio signals only (no light) for receipt by one or both ears of a user.

In embodiments, the visual display component may comprise a wearable visor or wearable glasses for placement over one or both eyes of a user, wherein said visual display component further comprises one or more lights, such as, for example, LED lights, that emit light and/or light pulses in various wavelengths of visible and/or other non-visible light from the electromagnetic spectrum in accordance with the corresponding electric light signal or light signal portion of the A/V signal and according to the wavelength frequency of the associated LED light triggered by the light signal(s) for receipt, perception and detection of the eye(s).

Generally, in various embodiments, the resulting sound (audio) transmitted from the audio transmission component (as per the audio signal(s) received thereby) and the resulting light or light pulses emitted from the visual display component (as per the light signal(s) received thereby) are intended for receipt by the ears and eyes, respectively. Alternative embodiments, however, use auriculotherapy to stimulate the auricle portion of the human ear. Auriculotherapy is a health care procedure in which stimulation of the auricle of the external ear is utilized to alleviate health conditions in other parts of the body. As such, various embodiments using auriculotherapy mildly stimulate the brain by emitting light and/or light pulses in various wavelengths of visible and/or other non-visible light from the electromagnetic spectrum in the audio transmission component (e.g., headphones) of the human interface device that produce tiny vibrations detected by the ear auricle. Trigger points in the auricle detect the emitted transmissions, which are known to directly balance the body's organs and systems. These are typically activated using acupuncture needles, but light frequencies and other stimulations are known to have the same effect.

The A/V files used by embodiments of the invention transmit various audio and/or light signals that have been encoded therein using proprietary algorithms to produce sound and/or light patterns that specifically stimulate a user's brain and synchronize the user's brainwaves without any effort by the user. Audio files, or the audio file portion of combined A/V files, may be encoded through novel algorithmic means for stimulation and synchronization of different human brainwaves (e.g., alpha, beta, theta, delta, gamma, etc.) through isochronic tones and and/or binaural beats. Light signals may be further transmitted via the visual files or the visual portion of a combined A/V file through similar encoding means to simultaneously supplement or compliment the audio signals to achieve the same effect in a user. The resulting audio and/or light signals received by the human interface device component(s) and the audio and light transmitted and emitted therefrom and received by the eyes and ears maximize neuroplasticity—the brain's ability to reorganize itself by forming new neural connections, resulting in greater brain flexibility and resiliency.

Through such methods, embodiments of the invention induce in users, among other desirable effects, a state of relaxation, creativity and intuitiveness leading to a heightened state of consciousness, reduction in physical and emotional pain, and creating a clearer sense of purpose.

### BRIEF DESCRIPTION OF THE DRAWINGS

FIG. 1 is a schematic layout of an embodiment of the invention.

FIG. 1A is a schematic layout of an alternative embodiment of the invention.

FIG. 2 is a perspective view of an embodiment of the human interface device portion of an embodiment of the invention.

FIG. 3 is another perspective view of an embodiment of the human interface device portion of an embodiment of the invention.

FIG. 4 is another perspective view of an embodiment of the human interface device portion of an embodiment of the invention.

FIG. 5 is another perspective view of an embodiment of the human interface device portion of an embodiment of the invention.

FIG. 6 is a perspective view of an embodiment of the human interface device portion of an embodiment of the invention.

FIG. 7 is another perspective view of an embodiment of the human interface device portion of an embodiment of the invention.

FIG. 8 is another perspective view of an embodiment of the human interface device portion of an embodiment of the invention.

FIG. 9A is a chart depicting a sample of encoding in an A/V file for a twenty (20) minute playback session.

FIG. 9B is a chart depicting an alternative sample of encoding in an A/V file for a ten (10) minute playback session.

The within description and illustrations of various embodiments of the invention are neither intended nor should be construed as being representative of the full extent and scope of the present invention. While particular embodiments of the invention are illustrated and described, singly and in combination, it will be apparent that various modifications and combinations of the invention detailed in the text and drawings can be made without departing from the spirit and scope of the invention. For example, references to materials of construction, methods of construction, specific dimensions, shapes, utilities or applications are also not intended to be limiting in any manner and other materials and dimensions could be substituted and remain within the spirit and scope of the invention. Accordingly, it is not intended that the invention be limited in any fashion. Rather, particular, detailed and exemplary embodiments are presented.

The images in the drawings are simplified for illustrative purposes and are not necessarily depicted to scale. To facilitate understanding, identical reference numerals are used, where possible, to designate substantially identical elements that are common to the figures, except that suffixes may be added, when appropriate, to differentiate such elements.

Although the invention herein has been described with reference to particular illustrative and exemplary physical embodiments thereof, as well as a methodology thereof, it is to be understood that the disclosed embodiments are merely illustrative of the principles and applications of the present invention. Therefore, numerous modifications may be made to the illustrative embodiments and other arrangements may be devised without departing from the spirit and scope of the present invention. It has been contemplated that features or steps of one embodiment may be incorporated in other embodiments of the invention without further recitation.

### DETAILED DESCRIPTION OF THE INVENTION

A more detailed description of the invention now follows.

In the following detailed description, reference is made to the accompanying drawings, which form a part hereof. In the drawings, the use of similar or the same symbols in different drawings typically indicates similar or identical items, unless context dictates otherwise.

The illustrative embodiments described in the detailed description, drawings, and claims are not meant to be limiting. Other embodiments may be utilized, and other changes may be made, without departing from the spirit or scope of the subject matter presented here.

One skilled in the art will recognize that the herein described components (e.g., operations), devices, objects, and the discussion accompanying them are used as examples for the sake of conceptual clarity and that various configuration modifications are contemplated. Consequently, as used herein, the specific exemplars set forth and the accompanying discussion are intended to be representative of the more general classes. In general, use of any specific exemplar is intended to be representative of its class, and the non-inclusion of specific components (e.g., operations), devices, and objects should not be taken as limiting.

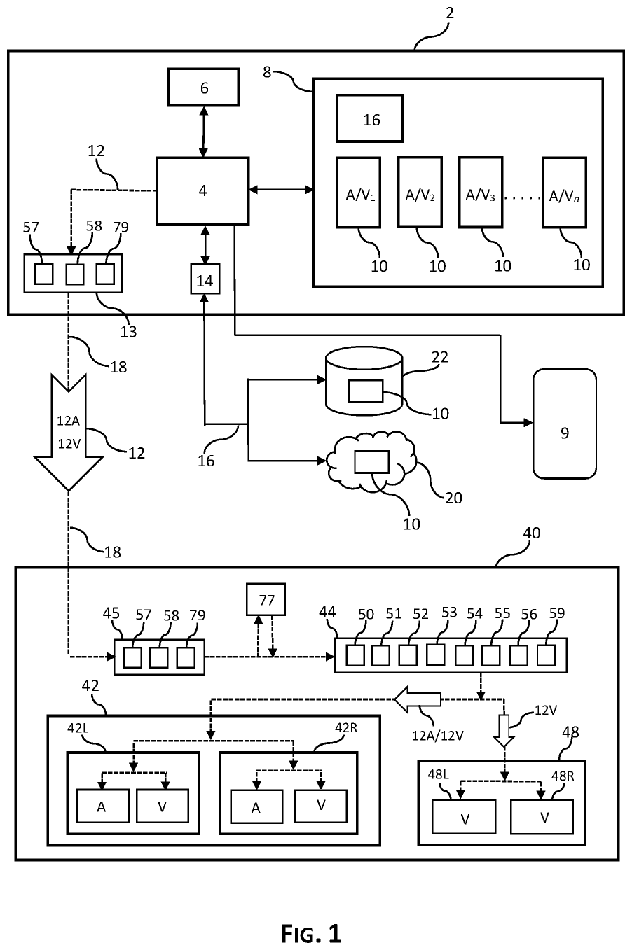

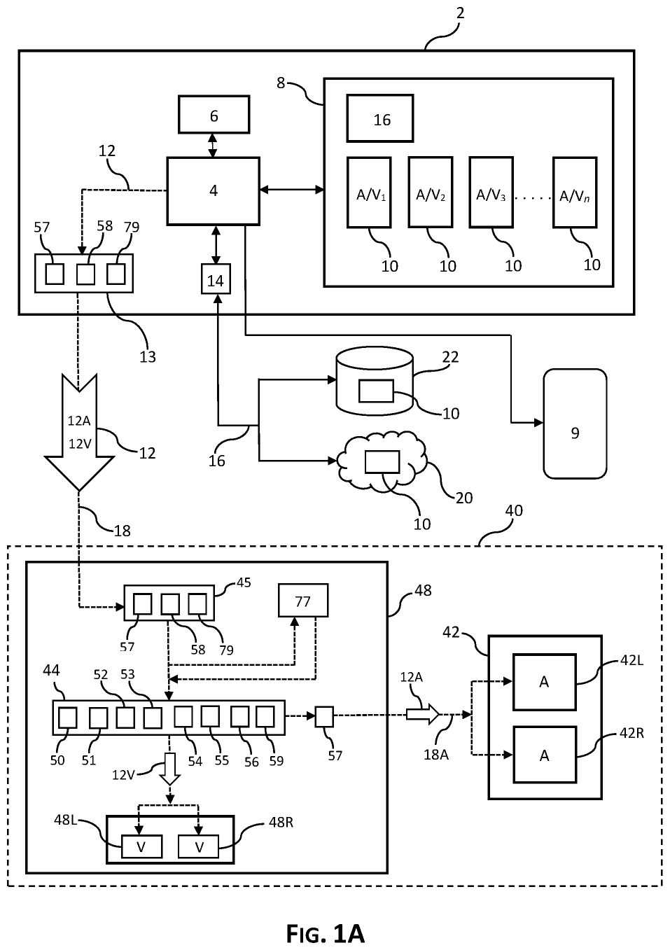

FIG. 1 is a schematic diagram of an embodiment of the invention. The embodiment depicted in FIG. 1 comprises an electronic playback device portion or component 2, said playback device 2 being capable of accessing and running (playing) one or more various pre-recorded digital audio and/or visual files 10 (i.e., A/V files) stored on playback device 2 or otherwise stored on such other external digital storage media 22, including storage media comprising the “cloud” of the Internet (e.g., servers in the “cloud”) 20 and accessible by playback device 2. Playback device 2 is functionally connected 18, either through a wired and/or wireless communication connection, to human interface device 40. In alternative embodiments, playback device 2 and human interface device 40 may be functionally integrated into a single component device. A/V files 10 played or otherwise accessed and streamed by playback device 2 are transmitted as electric (digital and/or analog) A/V signals 12 to human interface device 40 and converted to sound and/or light (or other non-visible electromagnetic waves) and experienced by a user as described in greater detail herein.

Continuing with FIG. 1, playback device 2 may further generally comprise a CPU 4 for accessing, decoding and converting A/V files 10 into A/V signals 12 for transmission to human interface device 40 through functional connection 18. A/V files 10 may be stored in a non-volatile memory or storage medium 8 in playback device 2, of which a user may add or delete any number of such A/V files 10 based on the user's preferences. CPU 4 of playback device 2 may be further functionally connected to a volatile memory 6 in the device 2 for decoding and converting A/V files 10 into A/V signals 12. Playback device 2 of FIG. 1 may be configured to store any number of A/V files 10 as determined by a user, said files 10 designated A/V₁, A/V₂, A/V₃ through A/Vₙ, with “n” being any number.

Playback device 2 of FIG. 1 may further access A/V files 10 stored external to the device, for example, on external digital storage media 22, such as, for example, an external hard drive or SSD drive, thumb drive, SD memory card or any such other digital storage media and devices commonly known and used for storing digital files, including multimedia files. Playback device 2 may digitally and functionally connect 16 to external digital storage media 22 through a connection interface 14, such as, for example, a USB port (USB 1.x, 2.x, 3.x, mini-USB, micro-USB, etc.), Apple® connector interface, network interface connector/controller (NIC), or any such other commonly known and used interface for digital electronic devices, including any proprietary connection interface. Playback device 2 of FIG. 1 may further access A/V files 10 stored on the internet, e.g., on servers in the “cloud” 20. Playback device 2 may digitally and functionally connect 16 to such servers 20 via any wired or wireless means and methods used by devices to functionally connect to the internet (e.g., network interface connector/controller (NIC) via cable, T1, T3, DLS, etc.), including wireless communication protocols and networks, e.g., WiFi, 4G, 5G, etc. In embodiments, users may access various databases of A/V files 10 stored in the cloud (internet) 20, select desired files for downloading or transmission to playback device 2, and store the selected A/V files 10 on playback device 2 for use at any time. Alternatively, A/V files 10 downloaded by a user from the cloud/internet 20 may be stored by a user in any available external storage device or media 22.

Continuing with FIG. 1, playback device 2 may further comprise playback software 16 designed for the purpose of playing multimedia files. Playback software 16 may be stored in storage 8 and accessed for execution by CPU 4 upon command by a user. A wide variety of general multimedia playback software 16 is widely available, generally at little to no cost and often as a functional component of a mobile device's operating system, and may offer a graphic user interface for display, access and use on a digital and/or electronic display 9 on or otherwise connected to playback device 2 to allow users to visually see and select specific A/V files 10 for playback. Such playback software 16 may further allow users to compile pre-determined “set lists” and incorporate other personally compiled meta data with a respective A/V file 10 for personal reference. Upon execution by a user, playback software 16 is accessed by CPU 4 and stored in memory 6 for immediate access and execution by CPU 4. Upon selection of a specific A/V file 10 by a user, software 16 accesses the respective A/V file 10 from storage 8 into memory 6 and plays the A/V file 10 by converting the digital file to its respective associated A/V signal(s) 12 for transmission to human interface device 40 via a functional electronic (digital and/or analog) connection 18.

In embodiments, software 16 may be an “app” downloaded and installed by a user on playback device 2. For example, playback device 2 may be a mobile device, such as a cell phone, running either the Apple® iOS® mobile device operating system or the Google® Android® mobile device operating system or any such other mobile device operating system that allows for downloading and installation of mobile “app” software programs. In such cases, the “app” may be proprietary (and, thus, be specifically directed for use with the associated A/V files) and available for free or for cost to download via the applicable operating system of the mobile device (playback device 2).

In embodiments, playback device 2 may take the form of any computerized device capable of playing multimedia files, including the streaming of files as discussed below. Examples include, but are not limited to, workstation computers, laptop computers, tablet computers or any hand-held device such as smart phones, cell phones, multimedia devices (iPods, MP3 players, etc.). Modern cell phones are particularly well-suited given their ease and convenience of connectivity to wireless communications networks, including the cloud/internet, generous and easy to use storage capabilities, relatively large, intuitive GUI displays, and user operability via touch screen technology. Modern cell phones further allow for readily available functional connectivity to human interface device 40 via Bluetooth, USB, input/output audio jack, 3.5 mm auxiliary jack, RCA A/V jack, and other wired and wireless technologies commonly known and used.

In various embodiments, A/V files 10 are not stored on playback device 2, or other storage means, such as external storage devices or media 22, but rather, are “streamed” to playback device 2 through an “app” on device 2 that connects via a functional digital network connection to an online platform or service, such as a website or other platform hosted on a server in the cloud or on the internet 20, and played by playback device 2 as portions of the A/V file 10 are received by the app running on the device from the online platform. Streaming is a technology used to deliver content to computers and mobile devices over the internet. Streaming transmits data—usually audio and video, but increasingly other kinds as well—as a continuous flow, which allows the recipients to begin to watch or listen almost immediately.

Streaming, in general, offers an alternative and expedient method to access internet-based content—in this case, A/V files 10. The key differences between downloading a file and streaming a file are generally directed towards 1) when a user can start using the content and 2) what happens to the content after the user is done with it. With downloading, a user generally must download the entire file before being able to use it. The downloaded file is stored on the user's device (in this case, generally, playback device 2 or external storage media 22) and generally may be accessed any number of times by a user, i.e., the downloaded data/content file is stored on a user's device until the user deletes it. Streaming, on the other hand, allows a user to start using the content before the entire file is downloaded. In addition, with streaming, the content files are automatically deleted after use, i.e., the files are not saved (stored) on a user's device. Users of streaming services, including downloaded and installed apps on the mobile device, may nonetheless compile personal “set lists,” favorites, and other sets of files, with meta data supplied by the user, in an online personal account available for use by the user.

Countless online streaming services currently exist for audio, video and other forms of multimedia. Online platforms such as Spotify®, Apple Music®, Pandora®, iHeartRadio®, etc., currently offer audio and music streaming for immediate play on devices connected to those platforms. Online platforms such as Netflix®, Hulu®, YouTube®, Amazon Prime Video®, etc., currently offer streaming of video content, such as movies, shows, sports and other entertainment for immediate play on devices connected to those platforms. In all such cases, various multimedia files—from audio to HD video—are selected by a user from a device connected to the online platform, the associated file of that content is then streamed to the user's device and the streaming file is played by the device as it is received. Technologies and protocols in this regard are widely known and available. The streaming service may be proprietary and utilize a proprietary app downloaded and installed on a user's playback device 2, such as, for example, a user's mobile device or cell phone.

With respect to embodiments of the invention, whether A/V files 10 are stored locally on or within playback device 2, stored externally in the cloud 20 or other storage device 22 for downloading, or streamed from the internet/cloud 20 directly to playback device 2 in accordance with commonly known and established methods and protocols, when in use, playback device 2 plays an A/V file 10 selected by a user. In each such case, upon playing an A/V file 10, playback device 2 converts the digital A/V file 10, and its various encoding, to A/V signals 12 (comprising audio signal(s) 12A and/or light signal(s) 12V) for transmission via connection 18 to human interface device 40 wherein said A/V signals 12 are converted to light and sound/audio for receipt, detection and perception by the eye(s) and ear(s) of a user.

A/V files 10 may comprise any commonly known audio/visual or multimedia file format that allows for, upon playing, the transmission of audio signals 12A and/or light signals 12V. In an embodiment, A/V file 10 may comprise an audio-based file, such as, for example, an MP3 file, that permits additional encoding of light signals 12V for the objectives discussed in greater detail, below. Such file formats include, but are expressly not limited to, .WEBM, MPG, .MP2, .MP3, .MPEG, .MPE, .MPV, .OGG, .MP4, .M4P, .M4V. Other suitable file formats include .DAT and MIDI. Any file format that allows for the simultaneous encoding, playing and transmission of both audio signals 12A and light signals 12V (electrical signals for LED lights, as discussed below) are suitable. In embodiments, light signals 12V may comprise audio signals 12A associated with the transmission of sound at certain frequencies, typically at those not detected by the human ear, where, when received by human interface device 40, are detected and interpreted as a signal for emitting light by light emission feature V, which may generally comprise LED lights 60. It is understood that all references to light signals 12V herein are to further comprise audio signals 12A of various predetermined frequencies that are capable of receipt, detection and interpretation by human interface device 40 and activating LED lights 60 in accordance with the signal. For example, a continuous signal at a predetermined frequency (Hz) would result in a continuous activation of LED lights 60 and a continuous emission of light therefrom. Pulses of such signals at the pre-determined frequency or frequencies (Hz), on the other hand, would create intermittent activation of LED lights 60, thereby resulting in pulsating light emitted therefrom. Only audio signals at the pre-determined frequency would activate LED lights 60.

Continuing with FIG. 1, in an embodiment, human interface device 40 may generally comprise an audio transmission component 42, such as a headphone set, that allows for the transmission of audio and/or the emission of light to one or more ears, and/or a visual display component 48 for the emission of light to one or more eyes. In embodiments, human interface device 40 may further comprise one or more signal controls or control panel 44 comprising one or more signal controls. Various signal controls may include, but are not limited to: electric on/off switch 50; light (LED) on/off control 51; audio volume up control 52; audio volume down control 53; audio balance (right ear/left ear) control 54; one or more misc. signal control(s) 55 desirable for the human interface device 40 and commonly used for modulating audio and/or light signals; light (LED) dimmer control 56 for light display component 48 of human interface device 40; and on/off indicator light 59. Human interface device 40 may further comprise one or more various signal connection interfaces 45, such as, but not limited to, input/output audio jack 57 such as, for example, a 3.5 mm auxiliary jack, (e.g., audio input from playback device 2); USB port 58 for connecting to playback device 2 and for charging a rechargeable battery 77 in human interface device 40 to electrically power the device and its various features and elements; and Bluetooth® connecting element/connection interface 79 for connecting to playback device 2 via Bluetooth® wireless technologies. Rechargeable battery 77 may comprise any commonly known and used rechargeable batteries for mobile electronic devices, such as, for example, lithium ion batteries.

Continuing with the embodiment of FIG. 1, A/V signals 12, comprising audio signal(s) 12A and/or light signal(s) 12V, are generated by playback device 2 and may be transmitted to human interface device 40 through an electronically functional connection 18, such as suitable wiring, cable, jacks, etc. or through any widely available and commonly used wireless device connection protocols, such as, for example, Bluetooth® technology. In the embodiment depicted in the schematic diagram of FIG. 1, human interface device 40 is functionally connected to playback device 2 through connection interface 13, such as, for example, a USB port (USB 1.x, 2.x, 3.x, mini-USB, micro-USB, etc.) 58, Apple® connector interface, RCA A/V jacks, micro-jacks, cellphone type audio jacks (3.5 mm auxiliary jack) (commonly used) 57, Bluetooth® wireless technologies 79, or any such other common interfaces, technologies and protocols known and used, wired or wireless, for connecting digital electronic devices, including any proprietary connection interface. A/V signals generated by playback device 2 may be further received by human interface device 40 through a similar (or different) corresponding functional connection interface 45, such as, for example, a USB port (USB 1.x, 2.x, 3.x, mini-USB, micro-USB, etc.) 58, Apple® connector interface, RCA A/V jacks, micro-jacks, cellphone type input/output audio jacks (3.5 mm auxiliary jack) (commonly used) 57, Bluetooth® wireless technologies 79, or any such other common interfaces, technologies and protocols known and used, wired or wireless, for connecting digital electronic devices, including any proprietary connection interface. Upon receipt by human interface device 40, A/V signals 12, comprising audio signal(s) 12A (digital and/or analog) and/or light signal(s) 12V (digital and/or analog), are thereafter transmitted to audio transmission component 42, visual display component 48 and/or to any other desired signal controls (see, e.g., control panel 44) of human interface device 40 through widely known and commonly used functional electrical connections such as, for example, wiring, cables, electronic circuitry, etc., whereupon A/V signals 12 are transmitted as sound/audio and/or light to the user and which may be controlled or modulated by the user via the various signal control functions previously discussed.

In an embodiment, audio transmission component 42 of human interface device 40 may be in the form of a headphone set and cover one or both ears of a user. The headphone set (audio transmission component) 42 covering the ear(s) may be comprised of one or more audio speaker(s) A for the transmission of sound to the ear(s) from the corresponding audio signal 12A or audio signal portion 12A of A/V signal 12. Headphones 42 covering the ear(s) may be further comprised of one or more light emission features V, such as, for example, LED lights 60, for the emission of light (generally, in the form of light pulses) to the auricle of the ear(s) wherein said light or light pulses are of various wavelength frequencies of visible and non-visible light in accordance with the LEDs used and the corresponding light signal 12V or light signal 12V portion of A/V signal 12. Referring to the embodiment depicted in the schematic diagram of FIG. 1, headphones (audio transmission component) 42 may be further comprised of a left channel portion 42L for covering or use with the left ear of a user and a right channel portion 42R for covering or use with the right ear of a user. In accordance with the above, each channel—for each ear—may be further comprised of one or more audio speaker(s) A for the transmission of sound to the respective ear, and one or more light emission elements V, such as, for example, LED lights 60, for the emission of light (light pulses), to the auricle of the right and/or left ear(s) wherein said light or light pulses are of various wavelength frequencies of visible and non-visible light in accordance with the LEDs used and the corresponding light signal 12V or light signal 12V portion of the A/V signal 12.

Continuing with the embodiment as depicted in the schematic diagram of FIG. 1, alternatively, left channel 42L, which covers or is otherwise used with the left ear of a user, and right channel 42R, which covers or is otherwise used with the right ear of a user, may be configured to only comprise one or more audio electroacoustic transducers (e.g., audio speakers) A or other electroacoustic converters for converting electrical audio signals into corresponding sounds, with no light or light emitting feature V in either or both channels, 42L, 42R. Alternatively, in various embodiments, human interface device 40 may only comprise a headphone (audio transmission component) 42 and no visual display component 48, and wherein headphones 42 only comprises one or more audio electroacoustic transducers (e.g., audio speakers) A or other electroacoustic converters for converting and transmitting electrical audio signals into corresponding audio for left channel 42L (left ear) and right channel 42R (right ear). Examples of such embodiments of audio transmission component 42 comprising only audio speakers A include, but are not limited to, common “ear buds” widely used with cell phones, wireless ear buds, such as, for example, Airpods® by Apple, Inc., and more traditional earphones or headphones such as those manufactured by Bose®, Sony®, Beats®, JBL®, etc. It is expressly understood that the scope of the invention is not limited to any specific type of audio transmission component 42 where that component is only comprised of audio speakers A for the transmission of sound to a user and does not further comprise any light or light emission feature V within said audio transmission component.

Continuing with the embodiment depicted in the schematic diagram of FIG. 1, left channel 42L (covering a user's left ear) comprises a light emission feature V, such as one or more LEDs 60, electrically connected to receive electrical (digital and/or analog) light signals 12V from playback device 2. Similarly, right channel 42R (covering a user's right ear) also comprises a light emission feature V, such as one or more LEDs 60, electrically connected to receive electrical (digital and/or analog) light signals 12V from playback device 2. Upon receipt of an electrical light signal 12V (such as, for example, an audio signal at a pre-determined frequency (Hz)—typically at the upper range of that detectable by the human ear), the one or more LEDs 60 of light emission feature(s) V are activated, thereby emitting light or pulses of light in accordance with the associated electrical light signal and according to the encoding parameters of the signal as discussed in greater detail herein.

In embodiments, LEDs 60 comprising light emission feature(s) V of audio transmission component 42 may be of different wavelengths (frequencies) thereby emitting visible light of different colors. Alternatively, LEDs 60 may emit non-visible light from the electromagnetic spectrum, such as infrared, UV or other non-visible light of various wavelength frequencies.

Continuing with the schematic diagram of FIG. 1, human interface device 40 is further comprised of visual display component 48. In embodiments, visual display component 48 may comprise a wearable visor or wearable glasses for placement over one or both eyes of a user, wherein said visual display component 48 further comprises one or more light emission features V, such as, for example, LED lights 60, that emit various wavelengths of visible light and/or other non-visible light from the electromagnetic spectrum (such as infrared radiation/light) in accordance with the corresponding electrical light signal or light signal portion of the A/V signal, and according to the wavelength frequency of the associated LED light triggered by the light signal, for receipt, perception and detection of the eye(s). Continuing with the embodiment depicted in the schematic diagram of FIG. 1, left channel 48L (covering a user's left eye) comprises a light emission feature V, such as one or more LEDs 60, electrically connected to receive electrical (digital and/or analog) light signals 12V from playback device 2. Similarly, right channel 48R (covering a user's right eye) also comprises a light emission feature V, such as one or more LEDs 60, electrically connected to receive electrical (digital and/or analog) light signals 12V comprising A/V signal 12. Upon receipt of an electrical light signal 12V, the one or more LEDs 60 of light emission feature(s) V of light display component 48 are activated, thereby emitting light or pulses of light in accordance with the associated electrical light signal and according to the encoding parameters of the signal as discussed in greater detail herein.

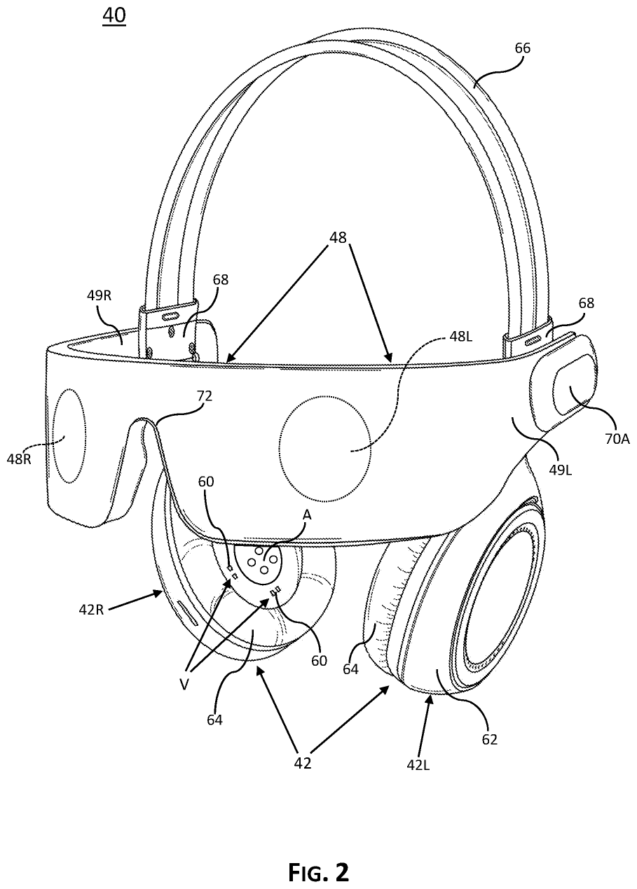

FIG. 2 is a perspective view of human interface device 40 of an embodiment of the invention. Human interface device 40 of FIG. 2 comprises both an audio transmission component 42 (in the form of a set of headphones) and a visual display component 48 (in the form of a visor) adjustably and detachably attached to each other to comprise the whole of human interface device 40.

Continuing with FIG. 2, visual display component 48 is a visor-type apparatus for covering the eyes of a user and is comprised of right channel or side 48R (depicted in phantom), which covers or is placed immediately in front of the right eye of a user, and left channel or side 48L (depicted in phantom), which covers or is placed immediately in front of the left eye of a user. Visual display component 48 is detachably and adjustably attached to human interface device head support strap 66, which, when worn by a user, said strap 66 crosses over the top or crown of the user's head to support human interface device 40. Support strap 66 is adjustably and detachably attached to visual display component 48 via attachment element 68, which allows visual display component 48 to be moved up and down in front of a user's eyes for proper vertical placement (by raising and lowering the visor feature on support strap 66), as well as for adjustment to move the visor of visual display component 48 closer to or further from the eyes by sliding the visor of visual display component 48 forwards and backwards for optimal effect and comfort. Visual display component 48 further comprises a nose bridge support feature 72 to allow visual display component 48 to rest on the bridge of a user's nose, much like eyeglasses would be supported.

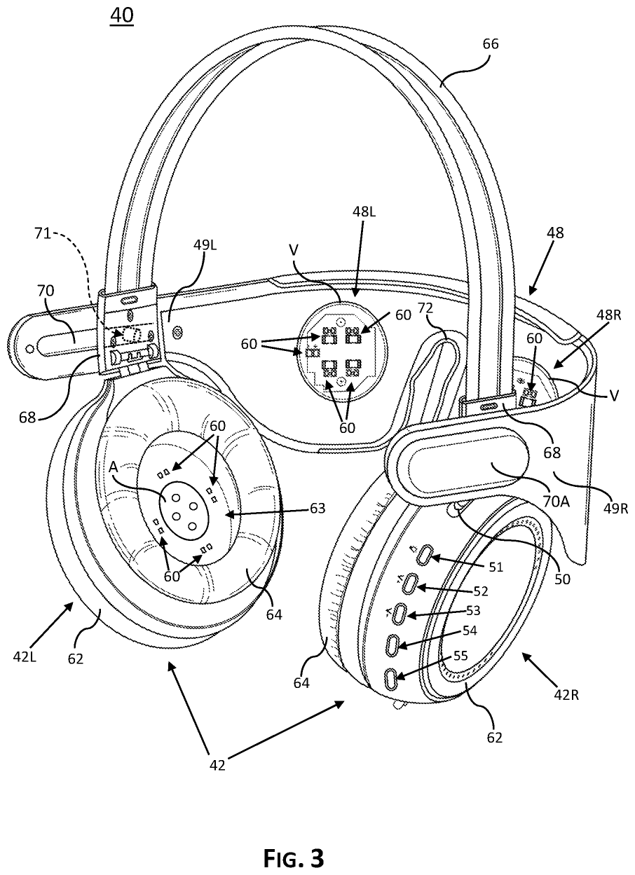

Continuing with FIG. 2, audio transmission component 42 is a headphone-type apparatus for covering the ears of a user and is comprised of right channel 42R, which covers or is placed immediately over the right ear of a user, and left channel 42L, which covers or is placed immediately over the left ear of a user. In the embodiment of FIG. 2, each channel is functionally equivalent to the other, although one or both channels may further comprise various signal controls or a control panel with various signal control functions, as discussed below with reference to FIG. 3. Channel components 42R, 42L of audio transmission component 42 are each comprised of an outer covering and housing portion 62 and an ear cushion 64 for comfortable placement over the ear and around the ear auricle. Configuration of cushion 64 around the ear-facing perimeter of housing 62, which is essentially circular to oval in shape, creates an inner void or space 63 within the perimeter of cushion 64 (see FIG. 3). Within covering and housing portion 62 of each channel 42R, 42L are one or more audio speakers A, for the transmission of audio or sound as previously described, and one or more light emission features V, in this case LEDs 60.

Human interface device 40 as depicted in FIG. 2 is worn on the head of a user and audio transmission component (headphones) 42 and visual display component (visor) 48 are adjusted accordingly to provide for a comfortable fit to the user. Alternatively, visual display component 48 may be removed via detachable attachment to attachment/adjustment element 68 and the audio transmission component (headphones) 42 may be worn without visual display component 48. To allow for detachable attachment and detachment, visual display component 48 further comprises an electric circuitry connection interface 90 (not depicted) for connection to a corresponding electric circuitry connection interface 92 (not depicted) in attachment/adjustment element 68, wherein electric connection interface 92 is functionally connected via wires, cables, circuitry, etc. to audio transmission component 42 and its corresponding playback device connection interface(s) 45, controls 50-56, 59 and/or control panel 44, and rechargeable battery 77, thereby creating an integrated, functional electrical connection between and among all electrical components comprising the embodiment. When visual display component 48 is properly attached to attachment/adjustment element 68, electric circuitry connection interface 90 and electric circuitry connection interface 92 are in proper alignment and contact, thereby creating a functional electric circuitry connection between visual display component 48 and audio transmission component 42 via the corresponding electric circuitry connection interfaces 90, 92.

FIG. 3 is another perspective view of the embodiment of human interface device 40 as depicted in FIG. 2. Human interface device 40 of FIG. 3 comprises both an audio transmission component 42 (again in the form of a set of headphones) and a visual display component 48 (again in the form of a visor) adjustably and detachably attached to each other to comprise the whole of human interface device 40.

Continuing with FIG. 3, audio transmission component 42 is a headphone-type apparatus (headphone set) for covering the ears of a user and is comprised of right channel 42R, which covers or is placed immediately over the right ear of a user, and left channel 42L, which covers or is placed immediately over the left ear of a user. In the embodiment of FIG. 3, each channel is functionally equivalent to the other, although right channel 42R, which covers the user's right ear, further comprises a plurality of signal controls 51, 52, 53, 54, 55 as previously discussed with reference to FIG. 1. It is understood that placement of signal controls is not limited to any particular component, feature or location of human interface device 40, including any audio transmission component channel, i.e., 42R or 42L. Channels 42R, 42L of audio transmission component 42 are each comprised of an outer covering and housing portion 62 and an ear cushion 64 for comfortable placement over the ear and around the ear auricle. Configuration of cushion 64 around the ear-facing perimeter of housing 62, which is essentially circular to oval in shape, creates an inner void or space 63 within the perimeter of cushion 64. Cushion 64 and inner space 63 allow the one or more speakers A and the one or more light emission features V of each channel component 42R, 42L to rest above—and not necessarily come in contact with—the ear of the user. Clearly depicted in the headphone comprising left channel 42L are one or more audio speakers A (one such speaker A is depicted in FIG. 3), for the transmission of audio or sound as previously described, and one or more video emission features V, in this case LEDs 60.

Continuing with the visual display component 48 of human interface device 40 of FIG. 3, depicted on the left inside portion of the visor comprising visual display component 48 is left channel 48L with left light emission feature V comprising one or more LED lights 60. Depicted on the right inside portion of the visor comprising visual display component 48 is right channel 48R with right light emission feature V comprising one or more LED lights 60.

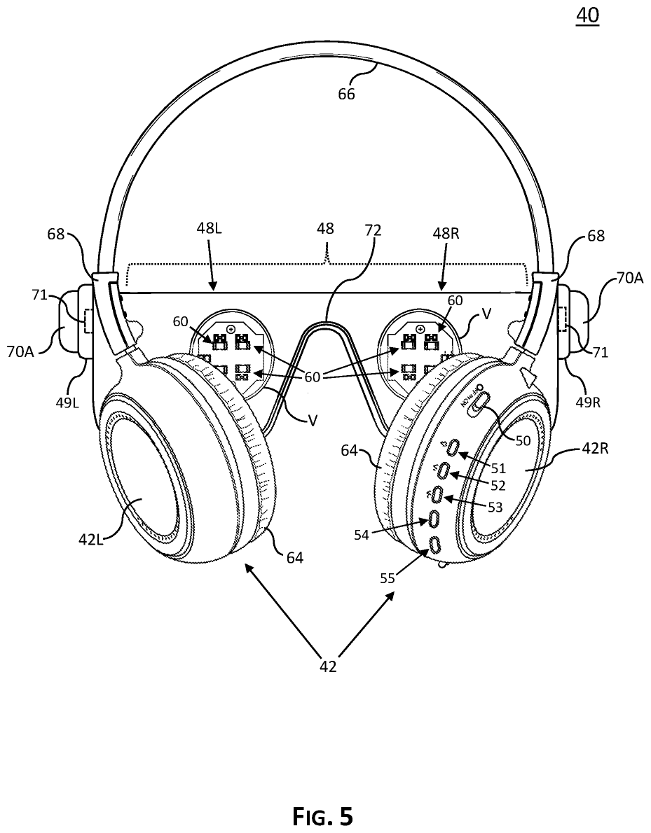

Visual display component (visor) 48 of human interface device 40 of FIG. 3 further comprises an adjustment slot 70 on the terminal end/side (arm) of visual display component arms 49L, 49R for adjustment of the visor 48 toward and away from a user's eyes by sliding the visor 48 forward and backwards along the alignment of slots 70. Inserted within adjustment slots 70 are corresponding slot elements 71 (featured in phantom on left terminal end 49L) that secure and hold visual display component 48 to adjustment/attachment elements 68 for proper and comfortable use. Visual display component 48 may be removed from human interface device 40 by accessing slot element 71 by removing or opening corresponding adjustment slot cover 70A (see also FIGS. 2, 5) to gain access to slot element 71. Slot element 71 may be a threaded bolt type element, or such other readily adjustable, securing and removable mechanical attachment means commonly used for such purposes, which a user may manually operate or manipulate to allow for the adjustment and/or attachment of the visual display component 48 from attachment/adjustment element 68, thereby removing it from human interface device 40. In such case, after removal of visual display component 48, human interface device 40 solely comprises audio transmission component 42, which in the embodiments of FIGS. 2 and 3 comprises a headphone headset featuring one or more speakers and one or more LEDs in each headphone channel, 42L, 42R.

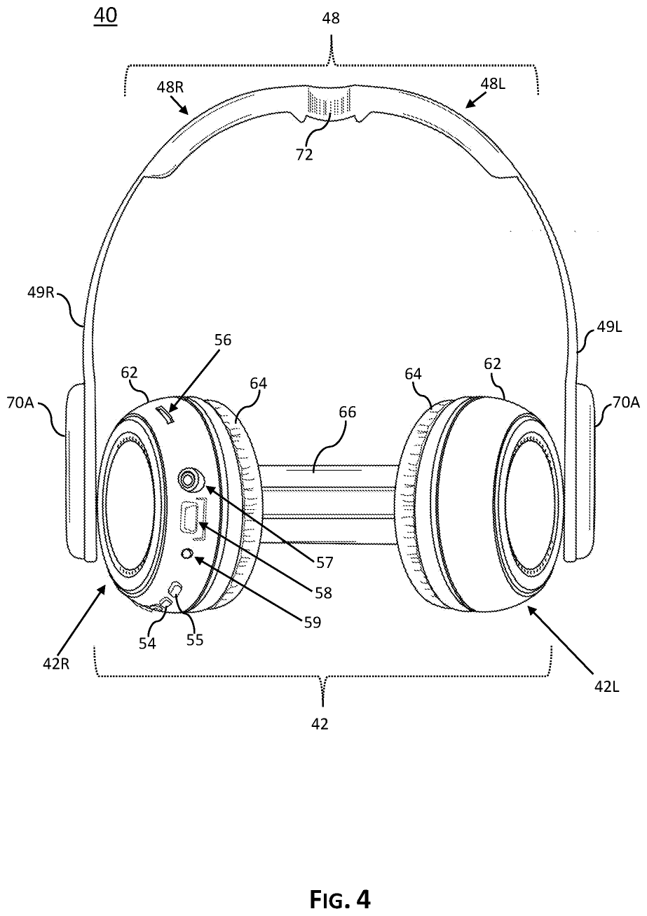

FIG. 4 is yet another perspective view of the embodiment of human interface device 40 as depicted in FIGS. 2 and 3. Human interface device 40 of FIG. 4 comprises both an audio transmission component 42 (in the form of a set of headphones) and a visual display component 48 adjustably and detachably attached or secured to each other to comprise the whole of human interface device 40. The perspective view of FIG. 4 is upward through the headphone (audio transmission component) 42 and towards the inner, head-facing side of support strap 66 with a view of the bottom periphery of visual display component 48, which in the depicted embodiment is in the form of a visor to cover the eyes. From this perspective, various signal controls, audio input jacks and digital interfaces of the embodiment of FIG. 4 are depicted. Specifically, light (LED) dimmer control 56, headphone input/output audio jack 57 (e.g., 3.5 mm auxiliary jack) (from playback device 2), USB connection port interface 58 and LED on/off indicator light are depicted. While the embodiments of FIGS. 2, 3 and 4 depict various signal controls, inputs, jacks, ports and/or interfaces, it is expressly understood that such controls, inputs, jacks, ports, interfaces, etc. are not limited to those depicted and in the configurations depicted in the drawings. Such controls may be located, place or configured in any fashion anywhere on human interface device 40.

FIG. 5 is yet another perspective view of the embodiment of human interface device 40 as depicted in FIGS. 2, 3 and 4. Human interface device 40 of FIG. 5 comprises both an audio transmission component 42 (again, in the form of a set of headphones) and a visual display component 48 (again, in the form of a visor) adjustably and detachably secured or attached to each other to comprise the whole of human interface device 40. The perspective view of FIG. 5 is through the back of the audio transmission component 42 (headphones) and towards the inner light emission features V of visual display component 48, which is in the form of a visor that covers the eyes. Further depicted in the embodiment of FIG. 5 is human interface device on/off switch 50 and signal controls 51, 52, 53, 54, 55. Also depicted (in phantom) in of FIG. 5 are corresponding slot elements 71 within adjustment slots 70 (not referenced) on left terminal end (visual display component arm) 49L and right terminal end (visual display component arm) 49R of visual display component (visor) 48 that secure and detachably adjust/attach visual display component 48 to adjustment/attachment elements 68 for proper and comfortable use. Visual display component 48 may be removed from human interface device 40 by accessing slot element 71 by removing or opening corresponding adjustment slot cover 70A (see also FIG. 2) to gain access to slot element 71. In addition, by accessing and then loosening slot elements 71, a user may slide visual display component (visor) 48 forward (away from) or back (towards) the user's face and eyes to achieve optimal placement of visual display component 48 for comfort and effect. Once a proper position is achieved, a user may tighten slot elements 71 to secure visual display component 48 in that configuration.

It is understood that while visual display component (visor) 48 may be adjusted in such manner, as long as it is attached to attachment/adjustment element 68, electric circuitry connection interface 90 (not depicted) remains functionally connected to electric circuitry connection interface 92 (not depicted) regardless of the manual adjustment of visual display component 48. In addition, audio transmission component 42 may likewise allow for adjustment with attachment/adjustment element 68 and also may comprise an electric circuitry connection interface 94 (not depicted) for connection to corresponding electric connection interface 92 (not depicted) in attachment/adjustment element 68. As such, visual display component 48 and audio transmission component 42 may be manually adjusted by a user for optimal comfort and fit and maintain an integrated, functional electrical connection between and among all electrical components comprising the embodiment regardless of the adjustment.

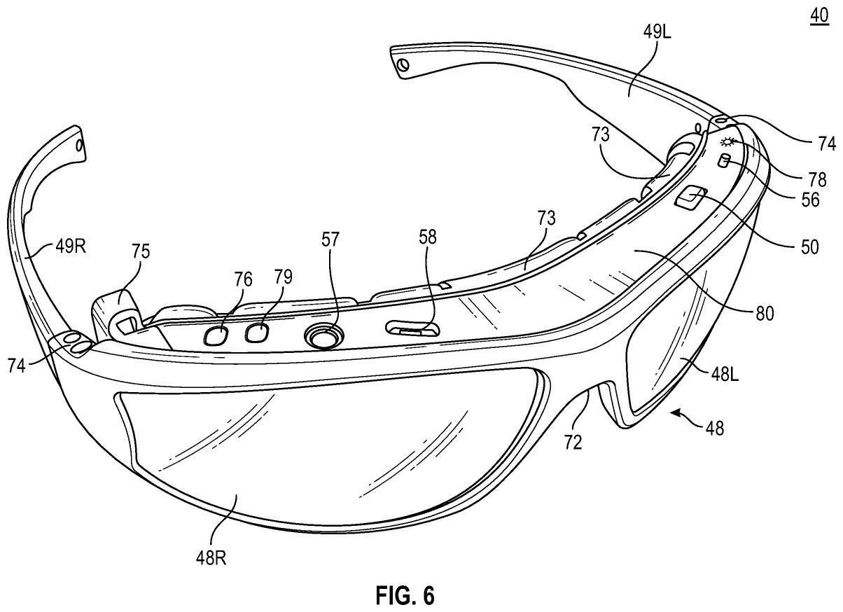

FIG. 6 is a perspective view of a further embodiment of human interface device 40. The embodiment of FIG. 6 is comprised primarily of a visual display component 48 in the general form of eyeglasses or goggles to be worn by a user with integrated controls, inputs, interfaces and ports to allow for a functional connection 18 (via wire or wirelessly) with playback device 2 (not pictured) and a functional connection 18A (see FIG. 1A) with optional separate audio transmission component 42 (not pictured).

Continuing with the embodiment of FIG. 6, visual display component 48 is comprised of right channel/side 48R that covers or is placed over a user's right eye during use and left channel/side 48L that covers or is placed over a user's left eye during use. Between right channel/side 48R and left channel/side 48L is nose bridge support element 72 for placement on the bridge of the nose of a user. Visual display component 48 is further comprised of right terminal side or arm 49R and left terminal side or arm 49L for placement over a user's right and left ears, respectively, to hold visual display component 48 firmly in place and to allow for adjustment by a user for proper fit and comfort. Visual display component 48 is further comprised of an upper bridge portion 80 that houses one or more device/signal controls, ports, inputs, interfaces, jacks and indicator lights. Continuing, visual display component 48 of FIG. 6 comprises an audio cable securing element 75, discussed below, a framework of cushions 73 on the interior side immediately adjacent to the eyes and face of a user, and hinges 74 to allow folding of the arms 49R, 49L when the device is not in use for proper storage and to avoid breakage. The embodiment of FIG. 6 is further comprised of an internal rechargeable battery 77 (not shown; see FIG. 1A) to provide electric power to the device as described below, said internal rechargeable battery 77 being located or housed in the upper bridge portion 80 of visual display component 48. It is understood that the embodiments of FIGS. 6-9 are not limited to the specific configuration of controls, battery, etc. and the embodiments may be alternatively configured.

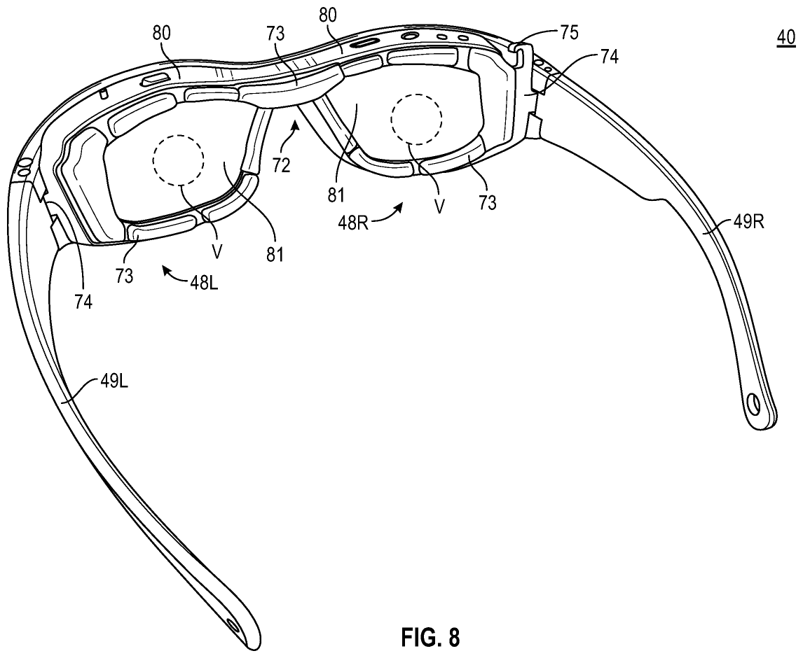

Continuing with the embodiment of FIG. 6, visual display component 48 is comprised of several device/signal controls, ports, inputs, interfaces, jacks and indicator lights as follows: on/off switch 50; light (LED) dimmer control 56; dimmer control indicator light 78; USB port 58; headphone input/output audio jack 57 (e.g., 3.5 mm auxiliary jack); Bluetooth® connecting element/interface 79; and charging indicator light 76. The audio and light features of the embodiment of FIG. 6 may be electrically powered and operated by an internal rechargeable battery 77 (not depicted) that is housed in the upper bridge portion 80 of visual display component 48. The rechargeable battery 77 may comprise any commonly known and used rechargeable batteries for mobile electronic devices, such as, for example, lithium ion batteries, and be recharged through the USB port 58 much like modern mobile devices—from cell phones to flashlights to “vape” products—are recharged. The appropriate USB cable may be inserted into USB port 58 and connected to playback device 2 for recharging or to any USB-powered port on a device, transformer, electrical plug-in, etc. While rechargeable battery 77 is being recharged through the USB port 58, charging indicator light 76 may emit light of a certain wavelength (color) and/or flash; when fully charged, charging indicator light 76 may emit light of a different wavelength (color) and/or cease flashing—any change in emission state to properly notify a user that battery 77 is fully charged. On/off switch 50 allows for powering on and off component 48 to conserve battery power resources. Light (LED) dimmer control 56 allows for brightening and dimming the LEDs 60 comprising the light emission features V discussed with reference to FIG. 8. Bluetooth® connecting element 79 allows for visual display component 48 to wirelessly connect with playback device 2 using Bluetooth® technology and protocols.

Continuing with the embodiment of FIG. 6, headphone input/output audio jack (e.g., 3.5 mm auxiliary jack) 57 allows a user to connect the user's personal audio transmission component 42 to the device. A user's audio transmission component 42 may generally be the headphones, earbuds, speakerphones, etc. associated with the user's personal playback device 2, such as a cell phone or other mobile device, which the user may connect to visual display component 48 via the playback device's Bluetooth® connecting option/feature, USB interface, etc. When visual display component 48 is connected to a Bluetooth® enabled playback device 2, A/V signals 12 are transmitted wirelessly to the Bluetooth® functionality 79 of visual display component 48. Light signals 12V or the light signal portion of the A/V signals 12 received by the Bluetooth® functionality 79 of visual display component 48 (or through any other functional connection, such as, but not limited to, USB, etc.) are transmitted via internal wiring, cables, circuitry, etc. to the light emission features V, discussed below, and audio signals or audio signal portion 12A of A/V signals 12 received by the Bluetooth® functionality 79 of visual display component 48 are transmitted via internal wiring, cables, circuitry, etc. to headphone input/output audio jack 57. When audio transmission component 42 is functionally connected via connection 18A (see FIG. 1A) to headphone input/output audio jack 57, such as through a cable, audio signals 12A are transmitted to audio transmission component 42 and the corresponding audio (sound) of audio signals 12A are transmitted from audio speakers A in audio transmission component 42 to the ears of a user. Audio cable securing element 75 allows for securing the cable of audio transmission component 42 (not depicted) to keep it from dangling loosely and to prevent accidental disconnection of the cable's jack (plug) from headphone input/output audio jack 57. In the embodiment of FIGS. 6-8, audio cable securing element 75 comprises a plastic clip in which a user may insert the audio transmission component cable (not depicted) for securing or detachable attachment during use.

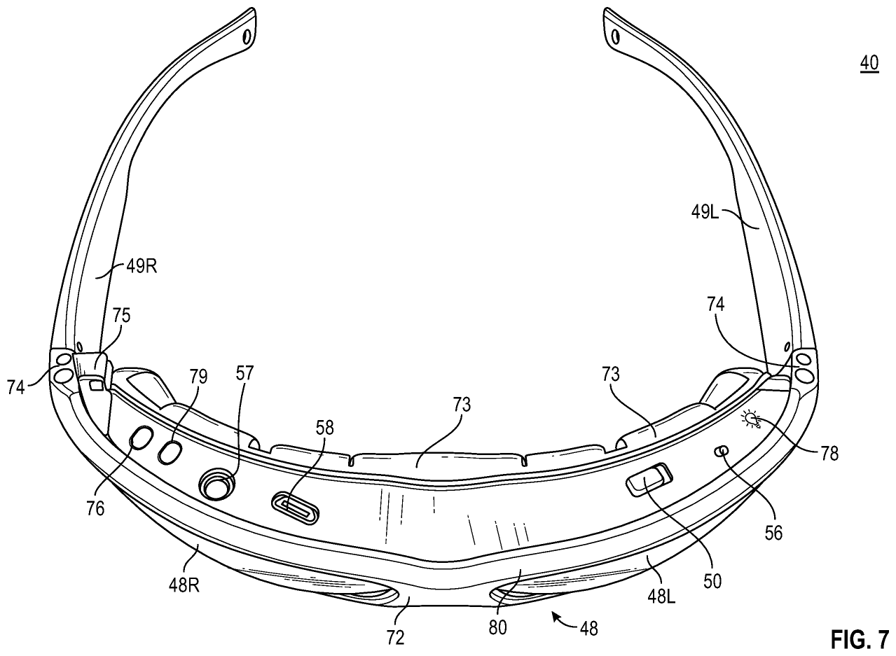

FIG. 7 is another perspective view of the embodiment of human interface device 40 of FIG. 6. The embodiment of FIG. 7 is comprised of primarily a visual display component 48 in the general form of eyeglasses or goggles to be worn by a user with integrated controls, inputs, interfaces, jacks and ports to allow for functional connection 18 (via wire or wirelessly) with playback device 2 (not pictured) and for functional connection 18A (via a cable, not depicted) with a separate audio transmission component 42 (not pictured).

Further depicted in the embodiment of FIG. 7 is the framework of eye/face cushions 73 on the interior side immediately facing the eyes and adjacent to the face of a user when the device is worn. The cushions provide a comfortable fit of visual display component 48 on the user's face and further blocks out external light and other visual distractions that may interfere with use of the invention. Note various spaces in the cushions to allow for the free movement of air and moisture in and out of the inner space (void) on the inside portion of visual display component 48 when being worn by a user.

FIG. 8 is another perspective view of the embodiment of human interface device 40 of FIGS. 6 and 7. The embodiment of FIG. 8 is primarily comprised of visual display component 48 in the general form of eyeglass or goggles to be worn by a user with integrated controls, inputs and ports to allow for functional connection 18 (via wire or wirelessly) with playback device 2 (not pictured) and for functional connection 18A (see FIG. 1A) with a separate audio transmission component 42 (not pictured). The perspective view of visual display component 48 of the embodiment of FIG. 8 is from that of a user that is using or about to use the visual display component 48 by placing it on the face, supported by nose and ears.

Continuing with the embodiment of FIG. 8, visual display component 48 is comprised of right channel/side 48R that covers or is placed over a user's right eye during use and left channel/side 48L that covers or is placed over a user's left eye during use. Between right channel/side 48R and left channel/side 48L is nose bridge support element 72 for placement on the bridge of the nose of a user. Visual display component 48 is further comprised of right terminal side or arm 49R and left terminal side or arm 49L for placement over a user's right and left ears, respectively, to hold the component firmly in place and to allow for adjustment by a user for proper fit and comfort. Visual display component 48 is further comprised of an upper bridge portion 80 that houses one or more device/signal controls, ports, inputs, interfaces, jacks and indicator lights. Continuing, visual display component 48 of FIG. 8 comprises an audio cable securing element 75, a framework of cushions 73 on the side immediately facing the eyes and adjacent to the face of a user, and hinges 74 to allow folding of the arms 49R, 49L when the device is not in use for proper storage and to avoid breakage. The embodiment of FIG. 8 is further comprised of an internal rechargeable battery 77 (not shown) to provide electric power to the device as described below, said internal rechargeable battery 77 being located or housed in the upper bridge portion 80 of visual display component 48.

Continuing with the embodiment of FIG. 8, right channel/side 48R and left channel/side 48L are each comprised of light emission features V, shown in phantom, which are located immediately behind translucent light filters 81. Light emission features V (in phantom) are comprised of one or more LED lights 60 (not depicted), which, when activated, emit light or light pulses in accordance with the light signals 12V received by component 48 from the playback device 2 and according to the wavelength frequency of the associated LED light triggered by the light signal(s) (see FIG. 1A). Translucent light filters 81 diffuse the light or light pulses emitted from LEDs 60, thereby creating a softer emission that covers the entire of the eyelid and surrounding orbital facial area for a more uniform and less harsh experience by the user. Alternatively, visual display component 48 may simply comprised light emission features V with one or more LEDs 60 in accordance with embodiments previously described.

FIG. 1A is a schematic diagram representative of the embodiments of FIGS. 6-8. In the embodiment of schematic diagram of FIG. 1A, human interface device 40 is comprised of separate visual display component 48 and audio transmission component 42, i.e., visual display component 48 and audio transmission component 42 are not functionally integrated or detachably attached into a single device or component those embodiments depicted in FIGS. 2-5. In the embodiment of schematic diagram of FIG. 1A, A/V signals 12 are received by visual display component 48 through a functional connection 18 in the manners previously described through various digital and/or electronic interfaces 45, such as, but not limited to: USB port (USB 1.x, 2.x, 3.x, mini-USB, micro-USB, etc.) 58 for connecting to playback device 2 and for charging a rechargeable battery 77 in human interface device 40 to electrically power the device and its various features and elements; Apple® connector interface; RCA A/V jacks; micro-jacks; cellphone type input/output audio jacks (e.g., 3.5 mm auxiliary jack) (commonly used) 57; and Bluetooth® connecting element/connection interface 79 for connecting to playback device 2 via Bluetooth® wireless technologies, or any such other common interfaces, technologies and protocols known and used, wired or wireless, for connecting digital electronic devices, including any proprietary connection interface. In embodiments, visual display component 48 may further comprise one or more signal controls or control panel 44 comprising one or more signal controls. Various signal controls may include, but are not limited to: electric on/off switch 50, which may be used to conserve electric power resources of rechargeable battery 77; light (LED) on/off control 51; audio volume up control 52; audio volume down control 53; audio balance (right ear/left ear) control 54; one or more misc. signal control(s) 55 desirable for the human interface device 40 and commonly used for modulating audio and/or light signals; light (LED) dimmer control 56 for light display component 48 of human interface device 40; and on/off indicator light 59. The embodiment of FIG. 1A may further comprise an internal rechargeable battery 77 to provide electric power to the device. Specifically, the audio and light features of the embodiment of FIG. 1A may be electrically powered and operated by internal rechargeable battery, such as, for example a lithium ion battery or any other rechargeable battery suitable for such purposes. The rechargeable battery 77 may be recharged through the USB port 58 much like modern mobile devices are recharged. The appropriate USB cable may be inserted into USB interface 58 and functionally connected via 18 to playback device 2 for recharging or to any USB-powered port on a device, transformer, electrical plug-in, etc. While rechargeable battery 77 is being recharged through the USB port 58, charging indicator light 76 may emit light of a certain wavelength (color) and/or flash; when fully charged, charging indicator light 76 may emit light of a different wavelength (color) and/or cease flashing—any change in emission state to properly notify a user that the battery 77 is fully charged. On/off switch 50 allows for powering on and off component 48 to conserve battery power resources.

Audio transmission component 42 of the embodiment of the schematic diagram of FIG. 1A is a separate component, such as headphones or earbuds, typically (but not necessarily) associated with a user's playback device. Audio transmission component 42 is functionally connected 18A to visual display component 48 via an audio signal interface, such as, for example, input/output audio jack (e.g., 3.5 mm auxiliary jack) 57, wherein the input cable to audio transmission component 42 may be functionally connected 18A to receive audio signals 12A from visual display component 48. In such embodiments, audio signal 12A or the audio signal portion 12A of A/V signals 12 are received by visual display component through any of its connection interfaces 45 as previously discussed. Said audio signal 12A or audio signal portion 12A of A/V signals 12 may pass through or be subject to any of the embodiment's signal controls 44 for manipulation or modulation by a user, e.g., audio volume, audio balance, etc. before passing or transmitting to input/output audio jack 57, whereupon said signal 12A is transmitted to audio transmission component 42 through the component's cable which is functionally connected 18A via a cable plug to input/output audio jack 57. The transmission wire or cable to the audio transmission component may be detachably secured to audio cable securing element 75, which may be an open cylindrical or curved plastic clip in which the cable is manually inserted for securing.

A/V files 10 comprising the system and played by playback device 2 are comprised of various encoding programming directed towards sounds, music, vocal instruction (spoken words, such as, for example, guided meditation), as well as specific frequency encoding to initiate specific light signals 12V. Various tracks are recorded and mixed into a single, overlaid A/V file. Tracks comprise various audio recordings, such as, but not limited to, isochronic tones, binaural beats, music, spoken words, and audio signals recorded at 19,000 Hz that emulate as light signals 12V. As A/V file 10, generally (but not limited to) an MP3 file, is played, the embedded 19,000 Hz signals—audio signals at or above the upper range of that detectable by the human ear—serve as light signals 12V and are transmitted to and received by light emission feature V, such as LEDs 60, which are activated and emit pulsating light in accordance with the coded signals. Isochronic tones, binaural tones, music and spoken words, all comprising audio signals 12A, are transmitted to and received by audio speaker(s) A of audio transmission component 42 and converted to audio waves for receipt by the ear. Only audio signals at or above 19,000 Hz (or at any other designated frequency and encoded accordingly in A/V file 10) activate LED lights 60.

Features of the audio signal portion 12A of A/V files 10 are those portions thereof directed towards producing and transmitting isochronic tones and binaural beats, which stimulate various brainwave activity. Isochronic tones are consistent regular beats of a single tone (the frequency at which the tone is presented is measured in hertz or Hz). Isochronic tones are regular beats of a single tone that are used alongside monaural beats and binaural beats to stimulate brainwave activity. At its simplest level, an isochronic tone is a tone that is being turned on and off rapidly; they are sharp, distinctive pulses of sound. The distinct and repetitive beat of isochronic tones produce an evoked potential, or evoked response, in the brain. Frequency following response (“FFR”) occurs when brainwaves become entrained (synchronized) with the frequency of an isochronic beat. As such, through FFR, embodiments of the invention, using isochronic tones, can “modulate” brainwave activity to enhance or improve mental states.

Binaural beats represent the auditory experience of an oscillating sound that occurs when two sounds with neighboring frequencies are presented to a user's left ear and right ear separately. When hearing the two frequencies simultaneously, the mismatch between the tones is interpreted by the brain as a new beat frequency. For example, when a 400 Hz sound frequency is delivered to the left ear, while a 405 Hz is delivered to the right ear, the brain processes and interprets the two frequencies as a 5 Hz frequency. Frequency following response (FFR) occurs at the 5 Hz frequency, producing brainwaves at the same rate of 5 Hz, thereby stimulating the brain and “modulating” brainwave activity. Research has shown that when a person listens to binaural beats for a recommended time, their levels of arousal change. Researchers believe these changes occur because the binaural beats activate specific systems within the brain. The four known categories of frequency pattern include the following:
Delta patterns. Binaural beats in the delta pattern are set at a frequency of between 0.1 and 4 Hz, which is associated with dreamless sleep.
Theta patterns. Binaural beats in the theta pattern are set at a frequency of between 4 and 8 Hz, which is associated with sleep in the rapid eye movement or REM phase, meditation, and creativity.
Alpha pattern. Binaural beats in the alpha pattern are set at a frequency of between 8 and 13 Hz, which may encourage relaxation.
Beta pattern. Binaural beats in the beta pattern are set at a frequency of between 14 Hz and 100 Hz, which may help promote concentration and alertness.

Light signal 12V, e.g., audio signal 12A or audio portion 12A of A/V signal 12 encoded within an MP3 file 10 at or above 19,000 Hz (or at any such other frequency intended for such purposes herein described) and transmitted upon playback of file 10, may be detected by an audio frequency analyzer “sniffer” chip (or any such other similar technology commonly known and available to detect high frequency audio signals) embedded in the circuitry of human interface device 40. When the analyzer chip detects audio signals at 19,000 Hz, a signal is transmitted to light emission feature V of visual display component 48 (or light emission feature V of audio transmission component 42 in various embodiments) which activate the LEDs, thereby emitting light in accordance with the time sequence of the signal. Embedded audio signals 12A at 19,000 Hz may also be specifically encoded to the right eye and then the left eye (48L and 48R), that is, light emission features V for the right and left eye may pulse light independently of each other in accordance with the “stereo” encoding of light signals 12V (19,000 Hz audio signals 12A) in A/V file 10. Such encoding is within the audio signal 12A of the respective MP3 file 10, wherein a user hears music, tones, beats, voice, etc. well within the range of human hearing. However, as 19,000 Hz audio is at the upper end of the human hearing range, and generally not detectable, such audio will not be perceived by the user. MP3 files may be recorded in such fashion using a multi-track audio mixing program—with the 19,000 Hz audio frequency portion comprising a single track of the mix—and then mixed down into stereo file for play back. As such, light signals 12V, audio signals 12A recorded at 19,000 Hz, are mixed into A/V file 10 but are only “heard,” i.e., detected, only by that portion of the various embodiments' circuit designed to “listen” for the 19,000 Hz frequency that controls the light duration and frequency. Generally, light emitted by LEDs 60 in visual display component 48 (or, in alternative embodiments, LEDs 60 in audio transmission component 42) flash or pulse in a general range of between 0.01 and 40 cycles per second (CPS) depending on the neuro training designed for the session and embedded in A/V file 10. More optimally, LEDs 60 will flash or pulse in the range of between 0.05 and 25 cycles per second. And, more optimally, LEDs 60 will flash or pulse in the range of between 0.1 and 20 cycles per second. A typical A/V file 10 will comprise evolving flash patterns throughout its playback in accordance with the desired effects sought by a user.

In embodiments, A/V files 10 are created to generate stereophonic audio and light, i.e., right channel 42R and left channel 42L for audio transmission component 42 and right channel 48R and left channel 48L for visual display component 48 (in alternative embodiments, right channel 42R and left channel 42L of audio transmission component 42 may also comprise light emission features V; see FIG. 1). To achieve desired results in brain stimulation and brain wave synchronization, right channel 42R and left channel 42L components of A/V file 10 (and, thus, right channel 48R and left channel 48L through use of, for example, 19,000 Hz signal encoding) are encoded differently to achieve specific objectives. In the recording process, A/V files 10 are split into separate recording tracks, which are subsequently mixed into a single audio A/V file 10, as follows: 1) music is generally recorded within a range of between 200 Hz and 800 Hz frequency output; more optimally, within a range of about 300 Hz and 700 Hz frequency output; more optimally, within a range of about 380 Hz and 600 Hz frequency output; and more optimally, within a range of about 432 Hz and 528 Hz frequency output, although music may be recorded at any frequency; 2) high frequency signals at 19,000 Hz (or any other predetermined frequency) are recorded (encoded) in A/V file 10 to emulate light signals 12V that activate LED lights 60 in visual display component 48 to emit pulsating light at various predetermined cycles per second (generally, between 0.05 and 25 cycles per second, as previously noted) that correspondingly shift brainwave activity, thereby achieving desired brainwave stimulation; 3) red and blue LEDs 60 in audio transmission component 42 are similarly encoded to flash or pulse at a frequency between 73 Hz and 4672 Hz (cycles per second) which changes at predetermined time intervals, such as, every 30 seconds to six minutes, more optimally every 1 to 4 minutes and more optimally, approximately every 2 minutes, to relax the brain and nervous system; and 4) A/V files may further comprise one or more tracks comprising spoken words (messages), such as, for example, a guided meditation, intended for full brain activation, wherein a message (spoken words) are alternatively transmitted to the left ear only, the right ear only and/or both ears. The resultant mix of these different frequencies of light, sound and vibration provide neuro-traing to the brain by creating a symphony of brainwaves that induce stimulation and entertainment.

Embodiments generally comprise a visual display component 42 and an audio transmission component 48 using LED lights 60 that emit light in various wavelengths that are both visible and/or not visible to the naked eye. In an embodiment, LED lights 60 are comprised of 470 nm wavelength (blue) and 633 nm wavelength (red) light, both of which are visible to the naked eye, although the scope of the invention is not limited to any particular light color or wavelength of visible light. Any wavelength of visible light may be suitable. Embodiments may also comprise visual display components and audio transmission components using LED lights 60 that emit 810 nm wavelength (near infra-red) light, generally non-visible to the naked eye. Light signals 12V (or audio signals 12A at 19,000 Hz, for example) are encoded in A/V files 10 to activate LED lights 60 in the above three (3) wavelengths—or any wavelength used—to pulse at the desired frequency (cycles per second or CPS) to create the desired brainwave stimulation. Generally, light emitted from LEDs 60 pulsing in visual display component 48 (for detection by a user's eyes) at 7-13 CPS stimulates alpha brainwave activity, 4-7 CPS for theta brainwave activity, 10-13 CPS for sensory motor rhythm and 0.5-4.0 CPS for delta brainwave training and stimulation.

Typical brainwave stimulation training by various embodiments are generally conducted in “sessions” of between ten (10) and twenty (20) minutes, which are determined by the length of time to play an entire A/V file 10, although sessions may be shorter or longer in duration. During a session, A/V file 10 is played by playback device 2 and A/V signals 12 are received by human interface device 40. The eyes of a user (and, thus, the user's brain) experiences pulsating visible and non-visible light from the visual display component 48. The ears of a user (and, thus, the user's brain) experiences audio, in the form of isochronic tones, binaural beats, music and voice, from the audio transmission component 42. In embodiments, the auricle of the user's ear (and, thus, the user's brain) experiences visible and non-visible light from the audio transmission component 42.

User's may benefit from daily sessions over an extended period of time. While users may undergo as many sessions as desired on a daily basis—there is no harm in multiple daily sessions—one (1) to three (3) sessions per day are optimal. Users are further recommended to practice daily sessions over a prolonged period of time typically in the order of several weeks in order to obtain maximum brainwave training benefit. Generally, five (5) weeks or more is optimal, with five (5) to ten (10) weeks being even more optimal. Users that continue daily sessions on a continuous, ongoing basis without interruption will only attain even greater benefit—sessions are akin to brain exercise and the brain benefits with continuous, extensive use.

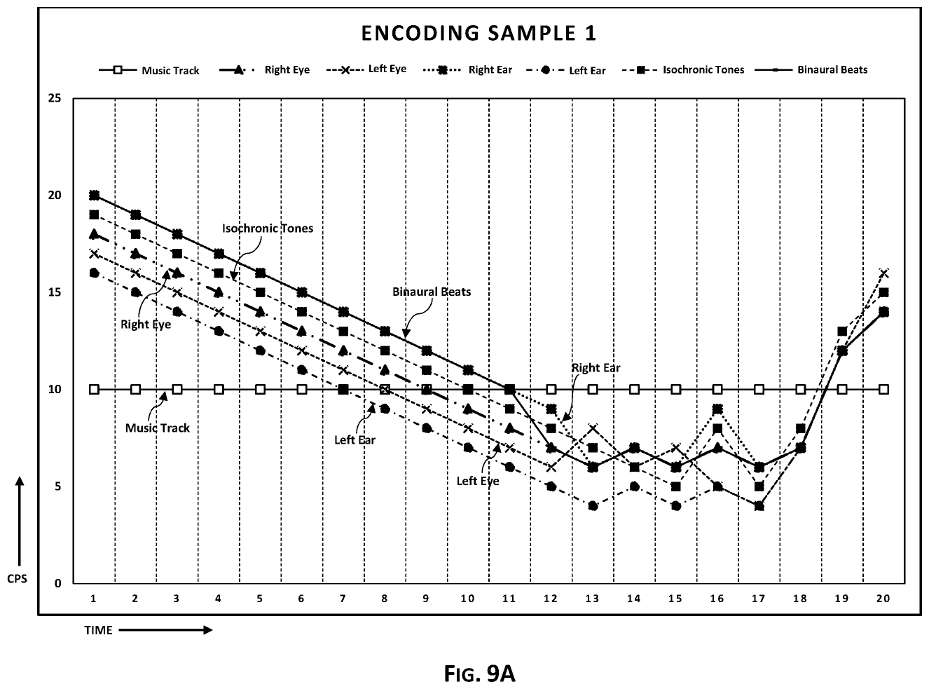

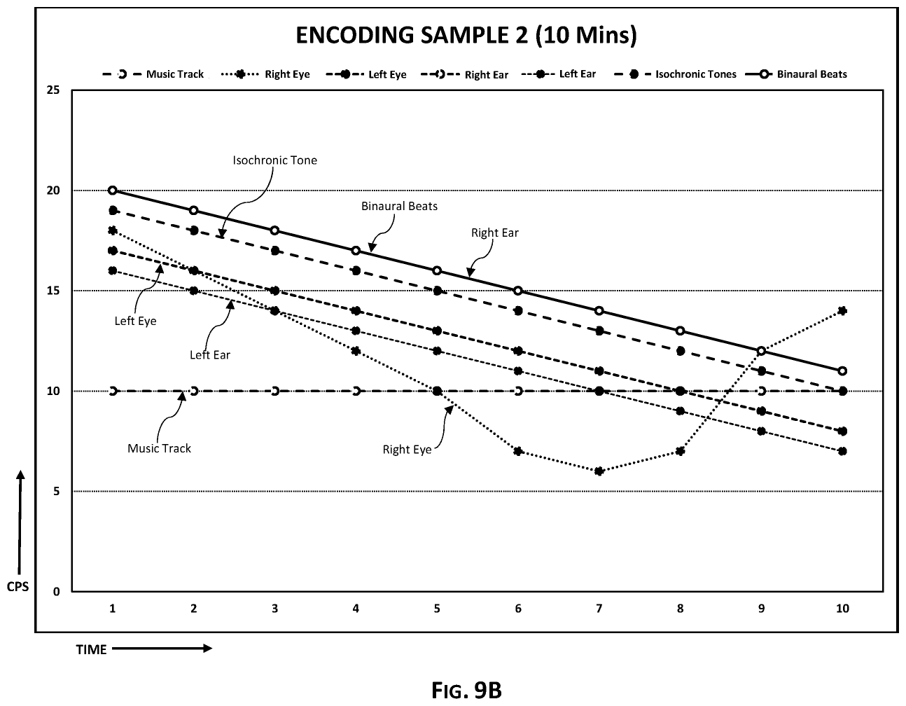

FIG. 9A is a chart depicting a sample encoding of an A/V file 10 for a twenty (20) minute session. The x-axis of the chart represents time in minutes. The y-axis of the chart represents cycles per second for the charted parameters. In the chart of FIG. 9A, data points are plotted for the following seven (7) parameters: 1) audible music (left and right ear), 2) left ear (light pulses in CPS), 3) right ear (light pulses in CPS), 4) left eye (light pulses in CPS), 5) right eye(light pulses in CPS), 6) isochronic tones (same for left and right ears as depicted in the chart) and 7) binaural beats (same for left and right ears as depicted in the chart).

FIG. 9B is a chart depicting a sample encoding of an A/V file 10 for a ten (10) minute session. The x-axis of the chart represents time in minutes. The y-axis of the chart represents cycles per second for the charted parameters. In the chart of FIG. 9A, data points are plotted for the following seven (7) parameters: 1) audible music (left and right ear), 2) left ear, 3) right ear, 4) left eye, 5) right eye, 6) isochronic tones, and 7) binaural beats.

Testing of various A/V files have produced significant positive benefits as a result of the invention's brainwave stimulation and synchronization effects. Following are two sample studies.

### Sample 1

In a 2018 6-week pilot study involving university students, the embodiment of FIGS. 2-5 was tested using an A/V file 10 specifically designed/encoded to enhance mood and the quality of sleep in the test subjects. The objective of the study was to investigate the effect of the invention on mood and sleep quality in preparation for a larger clinical trial to be conducted in 2019. The study protocol was reviewed and approved by the appropriate university ethics committee and institutional review board and informed consents were obtained from subjects prior to commencement of the study.

The study sample size and population consisted of seven (7) participants, four males and three females, between the ages of 20 and 58. While seven (7) participants is a small sample size, the purpose of the study was that of a pilot investigation to determine whether to pursue additional studies. Study participants were all university students who had no previous experience with the technology. In addition, potential candidates were excluded from participating in the study: individuals who had undergone previous surgeries, individuals who were making use of analgesics, anti-inflammatories or sleep aids within seven (7) days of the study start date and individuals with hearing disabilities.

The study involved using the invention for three (3) sessions per week for six (6) total weeks. At the conclusion of the study, subjects were evaluated using the following protocols to test the efficacy of the invention: Epworth Sleepiness Scale (“ESS”) for daytime sleepiness; Insomnia Severity Index (“ISI”); Pittsburgh Sleep Quality Index (“PSQI”); depression, anxiety and stress scale (“DASS-21”); and perceived stress scale (“EPS-10”). The following results were reported (all table data is the average of the participants' individual scores).

ESS (paired t-test). The ESS test was developed by Dr. Murray Johns for adults in 1990 and subsequently modified it slightly in 1997 to assess the “daytime sleepiness” of patients. The ESS is a self-administered questionnaire with 8 questions. Respondents are asked to rate, on a 4-point scale (0-3), their usual chances of dozing off or falling asleep while engaged in eight different activities. Most people engage in those activities at least occasionally, although not necessarily every day. The ESS score (the sum of 8 item scores, 0-3) can range from 0 to 24. The higher the ESS score, the higher that person's average sleep propensity in daily life (ASP), or their “daytime sleepiness.”

ESS scores are generally interpreted as follows: Individual scores of between 10 and 16 points indicates the individual has a high possibility of mild somnolence, while individual scores greater than 16 points indicates the individual has severe somnolence. A lower score, particular below 10 points, indicates the individual has a low propensity to sleep and relax. The results of the pilot study for the seven (7) test subjects for the ESS parameter were not statistically significant (NS). See Table 1, below.

ISI (paired t-test). The ISI is a 7-item self-report questionnaire assessing the nature, severity, and impact of insomnia. It was developed by Charles M. Morin, Ph.D., Professor of Psychology at the Université Laval in Quebec City, Canada. The ISI is one of the most widely used assessment instrument in clinical and observational studies of insomnia. Scores are classified as follows: 0-7=no clinically significant insomnia; 8-14=subthreshold insomnia; 15-21=clinical insomnia (moderate severity); and 22-28=clinical insomnia (severe). Although subjects in the pilot study generally observed positive benefits in ISI scores (i.e., lower scores), the results of the pilot study for the seven (7) test subjects for the ISI parameter were nonetheless not statistically significant (NS). See Table 2, below.

PSQI (paired t-test). The PSQI is a self-report questionnaire that assesses sleep quality over a 1-month time interval. The PSQI is an effective instrument used to measure the quality and patterns of sleep in the older adult. It differentiates “poor” from “good” sleep by measuring seven domains: subjective sleep quality, sleep latency, sleep duration, habitual sleep efficiency, sleep disturbances, use of sleep medication, and daytime dysfunction over the past month. Scoring of the answers is based on a 0 to 3 scale, whereby 3 reflects the negative extreme on the Likert Scale. The sub-scores are tallied, yielding a “global” score that can range from 0 to 21. A global score of 5 or more indicates poor sleep quality. The higher the score, the worse the quality: (0-4 points) good quality of sleep; (5 to 10 points) poor quality of sleep and (>10 points) presence of sleep disorder.

The results of the pilot study for the seven (7) test subjects for the PSQI parameter showed marked improvement in quality of sleep at a statistically significant level (*p<0.05 when compared to baseline evaluation). See Table 3, below.

DASS-21 (paired t-test). The DASS-21 test is a clinical assessment that measures the three related states (scales) of depression, anxiety and stress. Each of the three DASS-21 scales contains 7 items, divided into subscales with similar content. The depression scale assesses dysphoria, hopelessness, devaluation of life, self-deprecation, lack of interest/involvement, anhedonia and inertia. The anxiety scale assesses autonomic arousal, skeletal muscle effects, situational anxiety, and subjective experience of anxious affect. The stress scale is sensitive to levels of chronic nonspecific arousal. It assesses difficulty relaxing, nervous arousal, and being easily upset/agitated, irritable/over-reactive and impatient. Scores for depression, anxiety and stress are calculated by summing the scores for the relevant items. The following table summarizes score results for the level of severity for depression, anxiety and stress under the DASS-21 test (the higher the score, the greater the impairment in the evaluated category):

| Meaning | Depression | Anxiety | Stress |
|---|---|---|---|
| Normal | 0-9 | 0-7 | 0-14 |
| Mild | 10-13 | 8-9 | 15-18 |
| Moderate | 14-20 | 10-14 | 19-25 |
| Severe | 21-27 | 15-19 | 26-33 |
| Extremely severe | 28+ | 20+ | 34+ |

The results of the pilot study for the seven (7) test subjects for the ESS parameter were not statistically significant (NS). See Table 4, below.

PSS-10 (paired t-test). The PSS-10 is a ten (10) question, widely used psychological instrument for measuring the perception of stress in individual. It is a measure of the degree to which situations in one's life are appraised as stressful. Items were designed to tap how unpredictable, uncontrollable, and overloaded respondents find their lives. The scale also includes a number of direct queries about current levels of experienced stress. Individual scores on the PSS can range from 0 to 40 with higher scores indicating higher perceived stress. Scores ranging from 0-13 would be considered low stress. Scores ranging from 14-26 would be considered moderate stress. Scores ranging from 27-40 would be considered high perceived stress.

The results of the pilot study for the seven (7) test subjects for the PSS-10 parameter were not statistically significant (NS). See Table 5, below.

Conclusion. Participants in the study experienced a reduction in their ISI scores (data not statistically significant), PSQI scores (p<0.05), DASS-10 scores (data not statistically significant), PSS-10 (data not statistically significant). All participants reported feeling very relaxed during the sessions.

### Sample 2

In a second study, the embodiment of FIGS. 2-5 was tested on 100 subjects using an A/V file 10 specifically designed/encoded to enhance (increase) heart rate variability and parasympathetic activity and decrease stress and heart rates. The objective of the study was to investigate the effect of the invention on heart rate variability, parasympathetic activity, stress and heart rate after only a single 20-minute session. Participants were male and female. The following results were reported.

Increased Heart Rate Variability. Subjects experienced increased heart rate variability. Specifically, subjects experienced an average (mean) 21.8% increase in their heart rate variability (HRV) index. See Table 6, below. A low HRV value is associated with an increased risk of cardiovascular disease. In addition, subjects experienced an average (mean) 6.8% increase in their RRNN (RR normal-to-normal intervals; a marker of overall HRV activity). See Table 7, below.

Increased Parasympathetic Activity Markers. Subjects experienced increased parasympathetic activity markers. Specifically, subjects experienced an average (mean) 32.2% increase in their root mean square of the successive RR interval differences (“RMSSD”), a marker of parasympathetic activity (see Table 8, below); an average (mean) 50.6% increase in the number of pairs of successive NN (R-R) intervals that differ by more than 50 ms (“NN50”), again, a marker of parasympathetic activity (see Table 9, below); an average (mean) 51.6% increase in the proportion of NN50 divided by the total number of NN (R-R) intervals (“pNN50%”), again, a marker of parasympathetic activity (see Table 10, below); an average (mean) 37.1% increase in in their high frequency band (“HFnu”) index, an index of modulation of the parasympathetic branch of the autonomic nervous system (see Table 11, below); and an average (mean) 45.1% increase in their low frequency band (“Fnu”), a general indicator of aggregate modulation of both the sympathetic and parasympathetic branches of the autonomic nervous system (see Table 12, below). All test results were statistically significant.

Decreased Stress Index. Subjects experienced an average (mean) 38.5% decrease in their stress index score (see Table 13, below), a statistically significant result.

Decreased Heart Rate. Subjects experienced an average (mean) 6.2% decrease in their heart rate (see Table 14, below).

Conclusion. A single 20-minute session of using the embodiment of FIGS. 2-5 with an A/V file 10 specifically designed/encoded to enhance (increase) heart rate variability and parasympathetic activity and decrease stress and heart rates significantly (p<0.0001) increased heart rate variability and parasympathetic activity and significantly decreased (p<0.0001) stress index and heart rate in a clinical trial with 100 individuals.

Physical components and features comprising various embodiments of the invention may be comprised of any suitable materials required to achieve the intended purposes and objectives thereof as described herein. Playback device 2 may comprise any electronic and/or digital device to achieve the purposes and objectives disclosed. While not limited thereto, mobile devices and cell phones are particularly suited. Human interface device 40, as depicted in FIGS. 2-5 and FIGS. 6-8, may be generally comprised of plastics, resins, composites or any such other suitable materials generally available for such uses. Wires, cables, circuitry, switches, ports, interfaces, signal modulators, indicator lights, balance/volume controls, network and connectivity features, etc. are all well known, and embodiments herein are not limited to any particular configuration or otherwise. Translucent light filter 81 may be comprised of any suitable material to filter light to soften the glow, such as plastics or other suitable materials. Visual display component cushion 73 and headphone cushion 64 may be comprised of any such suitable foam material intended for such uses and may be further comprised of a thin cover made from plastic or other synthetic or nature material to protect the underlying foam cushion for body oils, perspiration, dirt, etc. Metal components may also be comprised of any suitable metal or alloy generally known and available for such uses.

While the invention has been disclosed in connection with embodiments shown and described in detail, various modifications and improvements thereon will become readily apparent to those skilled in the art. Accordingly, the spirit and scope of the present invention is not to be limited by the foregoing examples but is to be understood in the broadest sense allowable by law.

This disclosure of the various embodiments of the invention, with accompanying drawings, is neither intended nor should it be construed as being representative of the full extent and scope of the present invention. The images in the drawings are simplified for illustrative purposes and are not necessarily depicted to scale. To facilitate understanding, identical reference terms are used, where possible, to designate substantially identical elements that are common to the figures, except that suffixes may be added, when appropriate, to differentiate such elements.

Although the invention herein has been described with reference to particular illustrative embodiments thereof, it is to be understood that these embodiments are merely illustrative of the principles and applications of the present invention. Therefore, numerous modifications may be made to the illustrative embodiments and other arrangements may be devised without departing from the spirit and scope of the present invention. It has been contemplated that features or steps of one embodiment may be incorporated in other embodiments of the invention without further recitation.

Applicant claims the following:

1. A neuro-training device and system, comprising:
an electronic digital file playback component;
one or more electronic digital audio/visual (A/V) files for playback by the playback component; and
a human interface component functionally connected to the playback component,
wherein, the playback component is comprised of a memory, a CPU, a file storage, and a software for playing back the one or more A/V files, and
wherein, the one or more A/V files are encoded such that, upon play back, the A/V files generate one or more audio signals, one or more light signals, or a combination thereof, for transmission to the human interface component through the functional connection between the playback component and the human interface component, and
wherein, the human interface component is comprised of a visual display component further comprised of one or more light emission features for emitting pulses of light to be perceived by at least one eye of a user, and an audio transmission component further comprised of one or more audio speakers for the transmission of sound to be perceived by at least one ear of the user.

2. The neuro-training device and system of claim 1, wherein:
the one or more light emission features are comprised of LEDs.

3. The neuro-training device and system of claim 1, wherein:
the one or more audio signals are comprised of music, isochronic tones, binaural beats, or any combination thereof.

4. The neuro-training device and system of claim 1, wherein:
the one or more light signals are comprised of audio signals at frequencies above detection by human hearing.

5. The neuro-training device and system of claim 4, wherein:
the one or more light signals are comprised of audio signals at 19,0000 Hz.

6. The neuro-training device and system of claim 1, wherein:
the playback component is further functionally connected to the internet.

7. The neuro-training device and system of claim 6, wherein:
the playback component is further functionally connected to the internet.

8. The neuro-training device and system of claim 7, wherein:
the playback component is capable of downloading one or more A/V files stored on or more servers comprising the internet and storing the downloaded A/V files in the storage.

9. The neuro-training device and system of claim 8, wherein:
the playback component is further capable of streaming one or more A/V files stored the one or more servers comprising the internet for immediate playback of the one or more A/V files.

10. The neuro-training device and system of claim 1, wherein:
the A/V files are comprised of an MP3 file format.

11. The neuro-training device and system of claim 2, wherein:
the LEDs are comprised of wavelength frequencies of 470 nm, 633 nm, 810 nm, or any combination thereof.

12. The neuro-training device and system of claim 1, wherein:
the audio transmission component is further comprised of one or more light emission features for emitting light to an auricle of at least one ear of the user.

13. The neuro-training device and system of claim 12, wherein:
the light emission features of the audio transmission component are comprised of LEDs comprised of wavelength frequencies of 470 nm, 633 nm, 810 nm, or any combination thereof.

14. The neuro-training device and system of claim 1, wherein:
the visual display component of the human interface component is detachably attached to the audio transmission component as an integrated single device.

15. The neuro-training device and system of claim 1, wherein:
upon playback, the light signals generated thereby result in pulses of light of between 0.01 and 40 cycles per second (CPS), and more optimally of between 0.05 and 25 CPS, and most optimally of between 0.1 and 20 CPS.

16. The neuro-training device and system of claim 3, wherein:
upon playback, the isochronic tones and binaural beats generated thereby are between 0.1 and 40 cycles per second (CPS), and more optimally of between 1.0 and 25 CPS, and most optimally of between 5.0 and 20 CPS.

17. The neuro-training device and system of claim 13, wherein:
upon playback, the light signals generated thereby result in pulses of light emitted from the LEDs comprising the audio transmission component of between 73 Hz and 4672 Hz.
